SEQUENCE LISTING (58 sequences)

SEQ ID NO: 1 - DNA, 1521 bp, synthetic construct
  source 1..1521: mol_type = other DNA
atgaacaccattaatgaatacttgagtttagaagaattcgaagcaataatcttcggtaaccaaaaagtaactatctctgatgttgtcgtaaacagagttaacgaaagttttaacttcttaaaggaattttctggtaataaggttatatatggtgtaaacactggtttcggtccaatggctcaatacagaatcaaggaatctgatcaaatccaattgcaatacaatttgataagaagtcattcttcaggtactggtaaaccattatctcctgtttgtgctaaggctgcaatcttggcaagattgaacacattgtctttaggcaactcaggtgttcacccatctgttattaatttgatgtctgaattgataaacaaagacatcactcctttgatattcgaacatggtggtgttggtgcatctggtgacttggtccaattgtcccacttggccttagtattgataggtgaaggtgaagttttctataaaggtgaaagaagaccaacacctgaagtcttcgaaatcgaaggtttaaagcctatacaagtagaaatcagagaaggtttagctttgattaatggtacttctgtcatgacaggtataggtgttgtcaacgtataccatgctaagaaattgttggattggtcattgaagtccagttgtgccattaatgaattggttcaagcatatgatgaccatttctctgcagaattgaaccaaaccaagagacacaagggtcaacaagaaatcgcattgaagatgagacaaaatttgtccgatagtacattgatcagaaagagagaagaccacttatactcaggtgaaaacaccgaagaaattttcaaagaaaaggttcaagaatactactccttgagatgcgtcccacaaatcttgggtcctgtattggaaactattaataacgttgcctcaatcttggaagatgaattcaattccgctaacgataacccaatcatcgacgttaaaaatcaacatgtttatcacggtggtaacttccatggtgactacatttctttagaaatggacaaattgaagatagttatcacaaaattgaccatgttggctgaaagacaattgaactacttgttgaactcaaagattaacgaattgttgccacctttcgttaatttgggtacattgggttttaacttcggtatgcaaggtgttcaattcaccgccacttcaactacagctgaatcccaaatgttgagtaacccaatgtacgttcattccatccctaacaacaacgataaccaagacatcgtctctatgggtaccaactcagccgtcattacttccaaagtaatagaaaacgcattcgaagttttggccatcgaaatgatcacaattgtccaagctatcgattacttgggtcaaaaggacaagatctcttctgtttctaagaaatggtacgatgaaataagaaacataatcccaacctttaaggaagaccaagttatgtaccctttcgtacaaaaggttaaggatcatttgattaacaattaa

SEQ ID NO: 2 - DNA, 2093 bp, synthetic construct
  source 1..2093: mol_type = other DNA
atgaaaccagaagatttccgcgccagtacccaacgtcctttcaccggggaagagtatctgaaaagcctgcaggatggtcgcgagatctatatctatggcgagcgagtgaaagacgtcaccactcatccggcatttcgtaatgcggcagcgtctgttgcccagctgtacgacgcactgcacaaaccggagatgcaggactctctgtgttggaacaccgacaccggcagcggcggctatacccataaattcttccgcgtggcgaaaagtgccgacgacctgcgccagcaacgcgacgccatcgctgagtggtcacgcctgagctatggctggatgggccgtaccccagactacaaagccgctttcggttgcgcactgggcgcgaatccgggcttttacggtcagttcgagcagaacgcccgtaactggtacacccgtattcaggaaactggcctctactttaaccacgcgattgttaacccaccgatcgatcgtcatttgccgaccgataaagtgaaagacgtttacatcaagctggaaaaagagactgacgccgggattatcgtcagcggtgcgaaagtggttgccaccaactcggcgctgactcactacaacatgattggcttcggctcggcacaagtgatgggcgaaaacccggacttcgcactgatgttcgttgcgccaatggatgccgatggcgtgaaattaatctcccgcgcctcttatgagatggtcgcgggtgctaccggctcgccatacgactacccgctctccagccgcttcgatgagaacgatgcgattctggtgatggataacgtgctgattccatgggaaaacgtgctgatctaccgcgattttgatcgctgccgtcgctggacgatggaaggcggttttgcccgtatgtatccgctgcaagcctgtgtgcgcctggcagtgaaattagacttcattacggcactgctgaaaaaatcactcgaatgtaccggcaccctggagttccgtggtgtgcaggccgatctcggtgaagtggtagcgtggcgcaacaccttctgggcattgagtgactcgatgtgttcagaagcaacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaataatccgcagatcgaccagtatctggcgaagtatgtgcgcggttcgaacggtatggatcacgtccagcgcatcaagatcctcaaactgatgtgggatgctattggcagcgaatttggtggtcgtcacgaactgtatgaaatcaactactccggtagccaggatgagattcgcctgcagtgtctgcgccaggcacaaaactccggcaatatggacaagatgatggcgatggttgatcgctgcctgtcggaatacgaccaggacggctggactgtgccgcacctgcacaacaacgacgatatcaacatgctggataagctgctgaaataacgcagcaggaggttaagatgcaattagatgaacaacgcctgcgctttcgtgacgcgatggccagcctgtcggcagcggtaaatattatcaccaccgagggcgacgccggacaatgcgggattacggcaacggccgtctgctcggtcacggatacaccaccgtcgctgatggtgtgcattaacgccaacagtgcgatgaacccggtttttcagggcaacggcaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcatggacttatctactttaaacgccgtttccatccggtgatgctggaaatggaagctgcgatttaa

SEQ ID NO: 3 - DNA, 1293 bp, synthetic construct
  source 1..1293: mol_type = other DNA
atgaaaattgaagttaaagattcaactatgattaagcctagtgctgaaacaccaggtggttctttatggttgtcaaacttagatttgttatctccagctaattatcatactttatctgttcatttttattctcatgatggttctgataatttctttgatgctgcaggtttgaaagaatctttatcaagagctttagttgaattttatccatatgctggtagattgaaattgaatggtaatagattggaaattgattgtaataatgaaggtttgttattagttgaagctgaatgtgatggtgctttggatgaattaggagatttcgctcctagaccagaattgaacttaattccaaaagttgattattctagaggaatttctacatatccattaatggtatttcaattgactaggtttaaatgtggtggtgtagcattaggtgttgctaatgaacatcatctatctgatggtgttgcagcattacattttattaatacatgggctcatttgtcaagaggtgctccagcaccaactccattacctcattttgatagatcttctttgtctgctagaaatccacctcaacctcaattttctcatgctgaatatcaacctccaccaacattagaaaatccattgccacatacagatattgctcattctcgttttaaattgactagagatcaattgaattcattgaaaagtaagtttaaaactgcaccagcagatggtggtgctggtaaatcttattctacatttgaagttttagctggtcatatttggagatctgtctgtatagctagaggtttacctgaaggtcaagaaactaagttgcatattccatttgatggtagaggtagattacaattgccaccaggtttctttggtaatgctatctttttcgctactcctattgctacttgtggtgaaatagaatctaatagtttaaattatgctgttagaagagtttctgatggtgtttctagattggatgaagattatttgagatcttctatagattttctagaattacaagaagatatttctaaattagctcaaggtgctcattcattcagatgtccaaacttatgggtaattagttgggtttggttaccaatatatgaacctgattttggttggggtaaagctgtttatatgggtccatgggcagctccatttgaaggtaaatcatatttgttaccaaatcctgaaaaagatggttctttatttgtttctattactttacataaacaacatatggaaagatttgaaaaattgttttatgaaatttaa

SEQ ID NO: 4 - DNA, 942 bp, synthetic construct
  source 1..942: mol_type = other DNA
atggaagctattggtgttttgatgatgtgtccaatgtctacttatttggaacaagaattagataaaaggtttaaattgtttagatattggactcaaccagctcaaagagatttcttagcattacaagctgaatcaattagagctgttgttggtaattctaatgctggtgctgatgctgaattgattgatgcattaccaaaattagaaattgtttcttcttttagtgttggtttggataaagttgatttgataaaatgtgaagaaaaaggtgttagagttactaatacacctgatgttttgactgatgatgttgctgatttggctattggtttaattttggctgttttgagaagaatttgtgaatgtgataaatatgttagaagaggtgcttggaaatttggagattttaaattgacaacaaaattttctggtaaaagagttggtattattggtttaggtagaattggtttagctgttgctgaaagagctgaagcatttgattgtccaatatcttatttttctagatctaaaaaaccaaatactaattatacttattatggttcagttgttgaattagctagtaattctgatattttagtagttgcttgtccattgacaccagaaactacacatattattaatagagaagtcattgatgctttgggtccaaaaggtgttttgattaatatcggtagaggtccacatgttgatgaacctgaattggtttctgctttagttgaaggtagattaggtggtgcaggtttggatgtttttgaaagagaacctgaagttccagaaaaattatttggtttagaaaatgttgttttgttgccacatgttggtagtggtacagttgaaactagaaaagttatggctgatttggttgttggtaacttagaagctcatttttctggtaaaccattgttaactcctgttgtttaa

SEQ ID NO: 5 - DNA, 1635 bp, synthetic construct
  source 1..1635: mol_type = other DNA
atgggtgactgcgttgccccgaaagaggatctgatcttccgcagcaaactgccggacatttacattccaaagcatctgccgctgcatacctattgttttgagaacatcagcaaggttggcgacaagagctgtctgatcaacggcgcaaccggcgaaacctttacctacagccaggttgagctgctgtcccgtaaagttgccagcggcctgaacaagctgggcattcaacaaggtgataccattatgctgctgctgccgaactccccggagtactttttcgctttcctgggtgcgagctatcgcggtgcaatcagcactatggcgaacccattctttaccagcgcagaagtgatcaagcaactgaaagcgagccaagcgaagctgattatcacccaggcatgctatgttgacaaggttaaggactacgcagcggagaaaaacatccagatcatttgtattgacgatgcaccgcaggattgcctgcactttagcaagctgatggaagcggatgagagcgaaatgccggaagtggttattaacagcgatgatgtggtggcactgccgtacagctctggcaccaccggcctgccgaaaggcgttatgctgacccacaagggtctggttaccagcgttgcacaacaggtggatggtgataacccgaacctgtatatgcactccgaggatgttatgatctgcatcctgccactgttccatatctatagcctgaacgctgttctgtgttgtggtctgcgtgcgggcgttaccattctgatcatgcaaaagttcgacattgtgccgtttctggagctgattcagaagtataaggttaccattggtccgtttgttccgccgatcgtgctggccatcgcgaaaagcccggttgttgacaagtacgacctgtctagcgtgcgcaccgttatgagcggtgcagcgccgctgggtaaagagctggaggacgctgttcgtgcgaaattcccgaacgcgaagctgggtcaaggctatggcatgaccgaagccggtccggttctggcgatgtgtctggcgttcgccaaagagccgtatgagattaagtctggcgcatgcggtaccgttgtgcgtaacgccgagatgaaaatcgttgacccagaaaccaacgcgtctctgccgcgtaaccagcgtggtgagatttgcatccgtggtgatcagattatgaaaggttacctgaacgacccggaaagcacccgcaccaccatcgacgaagagggttggctgcacaccggtgacattggtttcatcgacgatgacgatgaactgttcattgttgatcgtctgaaagaaatcattaagtacaaaggttttcaagttgctccggcggagctggaagcactgctgctgacccacccgaccatcagcgatgccgcggtggttccgatgattgacgagaaagcgggtgaagtgccagtggcgtttgttgtgcgtaccaacggttttaccaccaccgaagaagaaatcaaacaatttgtgagcaaacaggttgtgttctacaaacgtatcttccgcgttttcttcgttgacgctattccgaaatccccgagcggcaagattctgcgtaaggatctgcgcgctcgtattgcgagcggcgacctgccgaagtaa

SEQ ID NO: 6 - DNA, 1053 bp, synthetic construct
  source 1..1053: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 7 - DNA, 2070 bp, synthetic construct
  source 1..2070: mol_type = other DNA
atgtttattgaaaccaacgtggcaaaaccggctagcacgaaagcgatgaatgccggctctgcaaaagcggccccggtcgaaccgttcgctacctatgcgcatagtcaggccaccaaaacggtgtccatcgatggccacacgatgaaagttggtgacgtggttgctgttgcgcgtcatggcgcgaaagttgaactggcagctagtgttgctggtccggtccgtgcgtccgtggattttaaagaaagcaaaaaacacacctcgatttatggcgtgaccacgggtttcggcggttcagccgatacccgtacgtcggacacggaagcactgcagatctctctgctggaacatcaactgtgcggctttctgccgaccgatgcgacgtacgagggtatgctgctggcggccatgccgattccgatcgtgcgtggtgcgatggcggtccgtgtgaacagctgtgttcgtggccactctggtgttcgcctggaagtcctgcagagctttgccgatttcattaatcgtggtctggttccgtgcgtcccgctgcgtggtaccatcagtgcatccggtgacctgtcaccgctgtcgtatattgctggcgcgatctgtggtcatccggatgttaaagtcttcgacaccgcagcttcaccgccgaccgttctgacgtcgccggaagcaattgcaaaatatggcctgaaaaccgtcaaactggcgagcaaagaaggcctgggtctggttaacggtacggcagtctctgcggcggcaggtgctctggcactgtacgatgccgaatgcctggcaatcatgagtcagaccaatacggtgctgaccgttgaagctctggacggccatgttggttcctttgcaccgttcattcaggaaatccgtccgcacgcgggccaaattgaagctgcgcgtaacatccgccatatgctgggcggttcaaaactggccgtgcacgaagaatcggaactgctggctgatcaggacgcgggtattctgcgtcaagatcgctacgccctgcgtaccagtgcacagtggatcggtccgcaactggaagccctgggtctggcacgccagcaaattgaaacggaactgaactccaccacggataatccgctgatcgacgtggaaggcggtatgtttcatcacggcggtaacttccaggcgatggcggtcaccagtgctatggattccgcgcgcattgtgctgcagaatctgggtaaactgtcatttgcacaagtgaccgaactgatcaactgcgaaatgaatcatggcctgccgtcgaacctggcgggtagcgaaccgtctaccaattatcattgtaaaggcctggatattcactgcggtgcctactgtgcagaactgggctttctggcgaacccgatgagcaatcatgttcagtctaccgaaatgcacaaccagagcgtgaacagcatggcgttcgcaagcgcacgtcgcacgatggaagcgaacgaagttctgagtctgctgctgggttcccagatgtattgtgctacccaagcgctggatctgcgcgtcatggaagtgaaatttaaaatggccattgtgaaactgctgaatgaaaccctgacgaaacattttgccgcattcctgaccccggaacagctggcgaaactgaacacccacgctgcgatcacgctgtacaaacgtctgaatcagaccccgtcatgggattcggcaccgcgctttgaagacgccgcaaaacatctggtgggcgttattatggatgcgctgatggttaacgatgacatcaccgacctgacgaatctgccgaaatggaagaaagaatttgccaaagaagcaggtaacctgtatcgtagcattctggtggctaccacggcggatggccgcaatgacctggaaccggccgaatatctgggtcagacccgtgccgtgtacgaagcagttcgcagcgaactgggcgtcaaagtgcgtcgcggtgatgttgcggaaggcaaaagcggtaaatctattggcagctctgtcgctaaaatcgtggaagcaatgcgtgacggtcgcctgatgggtgcagtgggtaaaatgttttaa

SEQ ID NO: 8 - DNA, 1425 bp, synthetic construct
  source 1..1425: mol_type = other DNA
atgaacatctctcgtcgtaaactgctgctgggtgttggcgcggcgggcgttctggcgggtggtgctgcgaccctggttccgatggttcgtcgtgatggtaaattcgttgaatctaaaagccgtgcgctgttcgtggaatctaccgaaggtgcgctgccgtccgaatctgacgttgttatcatcggcggcggtattcagggtatcatgaccgcaatcaacctggctgaacgtggtatgtctgtgaccatcctggaaaaaggcgaagtggcaggcgaacagagcggtcgtgcgtactctcagatcatcagctaccagacttctccggaaatcttcccgctgcaccactacggtaaaatcctgtggcgcggtatgaacgaaaaaatcggtgcggatacctcttaccgtacccagggtcgtgttgaagctctggcggatgaaaaagcgctggatcgtgctcaggaatggattaaaaccgctaaagaaaccgcgggcttcgatgtaccgctgaacacccgtatcatcaaaggcgaagaactgagcaaccgtctggttggcgcgcagaccccgtggaccgttgcggcgttcgaagaagatagcggcagcgttgatccggaaaccggtaccccgaccctggctcgttacgcgaaacagatcggcgtgaaaatctacacccactgcgctgttcgtggcatcgaaaccgcgggcggtaaaatcagcgacgttgttaccgaaaaaggtgcgattcgtacctccaacgttgtgctggcgggcggcatttggtctcgtctgttcatgggtaacatgggtgttgatctgccgaccctgaacgtttacctgtcccagcagcgcgtgtccggcgtgccgggtgcgccgcgtggtaacgttcacctgccgaacggcatccacttccgtgaacaggcggatggcacctacgcggttgcgccgcgcatcttcaccagctctatcgttaaagatagcttcctgctgggcccgaaattcatgcacctgctgggcggcggtgaactgccgctggaattcagcatcggcgaagatctctttaacagctttaaaatgccgaccagctggaaactggatgaaaaaagcccgttcgaacagtaccgtattgcaaccgcaacccagaacaccgaacacctggatgcggttttccagcgtatgaaaaccgaattcccggttttcgaaaaatcccagatcgttgaacgttggggtgcggtggtgtctccgaccttcgacgaactgccgatcatcagcgaagttaaagaatatccgggtctggttatcaacaccgcgaccgtttggggcatgaccgaaggtccggcggcgggtgaagtgaccgcggacatcgttaccggcaaaaaaccggttatcgatccgaccccgttcagcctggatcgtttcaaaaaataa

SEQ ID NO: 9 - DNA, 1686 bp, synthetic construct
  source 1..1686: mol_type = other DNA
atggcgccgcaggaacaggcggtttctcaggttatggaaaaacagtctaacaacaacaacagcgacgttatcttccgtagcaaactgccggatatttacatcccgaaccatctgtctctgcacgattacatctttcagaacatttctgaatttgcgactaaaccgtgcctgattaacggcccgaccggtcacgtgtacacctacagcgatgttcacgtgatcagccgccagattgcggccaacttccacaaactgggcgtgaaccagaacgatgttgtgatgctgctgctgccgaactgcccggaatttgttctgtctttcctggcggcaagctttcgtggtgctaccgcaaccgcggctaacccgttcttcaccccggcggaaatcgctaaacaggcaaaagcttctaacactaaactgatcatcactgaagcgcgctatgttgataaaatcaaaccgctgcagaacgatgacggtgttgttattgtgtgcatcgatgataacgaatccgtgccaatcccggaaggctgcctgcgtttcaccgaactgacccagtccaccaccgaagcgtccgaagtgattgatagcgtggaaatttctccggatgatgtagtggcgctgccgtactctagcggtactaccggtctgccgaaaggtgttatgctgacccataaaggtctggttacttccgttgcacagcaggttgatggtgaaaacccgaacctgtacttccactctgatgatgtgatcctgtgcgttctgccgatgttccacatctacgcactgaactctattatgctgtgtggcctgcgtgtaggtgctgcaattttgatcatgccgaaattcgaaatcaacctgttgctggaactgatccagcgttgcaaagtgaccgttgcgccgatggttccgccgatcgttctggcaattgctaaaagctctgaaaccgaaaaatacgacctgagctctatccgcgttgttaaaagcggtgcggcaccgctgggtaaagaactggaagatgctgttaacgctaaattcccgaacgcgaaactgggccagggttacggcatgaccgaagctggtccggtgctggcgatgagcctgggcttcgctaaagaaccgtttccggttaaatccggcgcatgtggtaccgtggttcgtaacgctgaaatgaaaatcgttgatccggataccggtgatagcctgtctcgtaaccagccgggtgaaatttgcattcgtggtcatcagatcatgaaaggctatctgaacaatccggcggcgaccgcggaaactatcgataaagatggctggctgcacaccggtgacatcggtctgatcgatgacgatgatgaactgtttatcgtagaccgtctgaaagaactgattaaatacaaaggcttccaggtggcgccggctgaactggaagctctgctgatcggccacccggatatcaccgatgttgcggtggttgcgatgaaagaagaagcggcgggtgaagttccggttgcgttcgttgttaaatctaaagactctgaactgagcgaagatgatgttaaacagttcgtgagcaaacaggttgtgttttataaacgtattaacaaagttttcttcaccgaaagcatcccgaaagcgccgtctggcaaaatcctgcgtaaagacctgcgtgcgaaactggcgaacggcctgtaa

SEQ ID NO: 10 - DNA, 882 bp, synthetic construct
  source 1..882: mol_type = other DNA
atgaccgttttcaaacacatcgcgatcatcggcctgggtctgatcggcagcagcgctgcgcgtgctaccaaagcgtactgcccggatgttaccgtgagcctgtacgataaaagcgaattcgtttgcgatcgtgcgcgtgcgctgaacctgggtgataacgtgaccgacgatatccaggatgcggttcgtgaagcggatctggttctgctgtgcgttccggttcgcgcgatgggtatcgttgcggcggcgatggcgccggcgctgaaaaaagatgttatcatctgcgataccggttctgttaaagtttctgttatcaaaaccctgcaggacaacctgccgaaccacatcattgttccgagccacccgctggcgggtaccgaaaacaacggtccggatgcaggcttcgcggaactgttccaggatcacccggttatcctgaccccggatgcgcacaccccggcgcaggcgatcgcgtacatcgcggattactgggaagaaatcggcggccgtattaacctgatgagcgcggaacaccacgatcacgttctggcgctgacctctcacctgccgcacgttatcgcgtaccagctgatcggcatggtttctggctacgaaaagaaatctcgtaccccgatcatgcgttacagcgcgggttctttccgtgatgcgacccgtgttgcggcgtctgaaccgcgtctgtggcaggacatcatgctggaaaacgcgccggctctgctgccggttctggatcacttcatcgctgatctgaaaaaactgcgtaccgcgatcgcgtcccaggatgaagattacctgctggaacacttcaaagaatctcagaaagcgcgtctggcgctgaaaaccgatcacgatatccacccgtaa

SEQ ID NO: 11 - DNA, 999 bp, synthetic construct
  source 1..999: mol_type = other DNA
atgaaaatcatcgcgtacgcggttcgtgatgatgaacgtccgttcttcgatacctggatgaaagaaaacccggatgttgaagttaaactggttccggaactgctgaccgaagataacgtggatctggcgaaaggtttcgatggcgcggatgttgcgcagcagaaagattataccgcggaagttctgaacaaactggcggatgaaggtgtgaaaaacatctctctgcgtaacgttggtgttgataacctggatgttccgaccgttaaagcgcgtggcctgaacatcagcaacgttccggcgtactctccgaacgcgatcgcggaactgtccgttacccagctgatgcagctgctgcgtcagaccccgctgttcaacaaaaaactggctaaacaggatttccgttgggcgccggacatcgcgaaagaactgaacaccatgaccgttggtgttatcggcaccggtcgtatcggtcgcgcggcgatcgatatcttcaaaggtttcggtgcgaaagttatcggttatgatgtttatcgtaacgcagaactggaaaaagaaggcatgtacgttgacaccctggatgaactgtacgcgcaggcggatgttatcaccctgcacgttccggcgctgaaagataactatcacatgctgaacgcggatgcgttcagcaaaatgaaagatggcgcgtacatcctgaacttcgcgcgtggcaccctgatcgatagcgaagatctgatcaaagcactggatagcggcaaagttgctggcgcggcgctggatacctatgaatatgaaaccaaaatcttcaacaaagatctggaaggtcagaccatcgatgataaagttttcatgaacctgttcaaccgtgataacgttctgatcaccccgcacaccgcgttctacaccgaaaccgcggttcacaacatggtgcacgttagcatgaacagcaacaaacagttcatcgaaaccggcaaagcggatacccaggttaaattcgattaa

SEQ ID NO: 12 - protein, 544 aa, synthetic construct
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASQAKLIITQACYVDKVKDYAAEKNIQIICIDDAPQDCLHFSKLMEADESEMPEVVINSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRARIASGDLPK

SEQ ID NO: 13 - protein, 561 aa, synthetic construct
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 14 - protein, 350 aa, synthetic construct
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLNMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 15 - protein, 293 aa, synthetic construct
MTVFKHIAIIGLGLIGSSAARATKAYCPDVTVSLYDKSEFVCDRARALNLGDNVTDDIQDAVREADLVLLCVPVRAMGIVAAAMAPALKKDVIICDTGSVKVSVIKTLQDNLPNHIIVPSHPLAGTENNGPDAGFAELFQDHPVILTPDAHTPAQAIAYIADYWEEIGGRINLMSAEHHDHVLALTSHLPHVIAYQLIGMVSGYEKKSRTPIMRYSAGSFRDATRVAASEPRLWQDIMLENAPALLPVLDHFIADLKKLRTAIASQDEDYLLEHFKESQKARLALKTDHDIHP

SEQ ID NO: 16 - protein, 506 aa, synthetic construct
MNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 17 - protein, 520 aa, synthetic construct
MKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLK

SEQ ID NO: 18 - protein, 170 aa, synthetic construct
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 19 - protein, 430 aa, synthetic construct
MKIEVKDSTMIKPSAETPGGSLWLSNLDLLSPANYHTLSVHFYSHDGSDNFFDAAGLKESLSRALVEFYPYAGRLKLNGNRLEIDCNNEGLLLVEAECDGALDELGDFAPRPELNLIPKVDYSRGISTYPLMVFQLTRFKCGGVALGVANEHHLSDGVAALHFINTWAHLSRGAPAPTPLPHFDRSSLSARNPPQPQFSHAEYQPPPTLENPLPHTDIAHSRFKLTRDQLNSLKSKFKTAPADGGAGKSYSTFEVLAGHIWRSVCIARGLPEGQETKLHIPFDGRGRLQLPPGFFGNAIFFATPIATCGEIESNSLNYAVRRVSDGVSRLDEDYLRSSIDFLELQEDISKLAQGAHSFRCPNLWVISWVWLPIYEPDFGWGKAVYMGPWAAPFEGKSYLLPNPEKDGSLFVSITLHKQHMERFEKLFYEI

SEQ ID NO: 20 - protein, 313 aa, synthetic construct
MEAIGVLMMCPMSTYLEQELDKRFKLFRYWTQPAQRDFLALQAESIRAVVGNSNAGADAELIDALPKLEIVSSFSVGLDKVDLIKCEEKGVRVTNTPDVLTDDVADLAIGLILAVLRRICECDKYVRRGAWKFGDFKLTTKFSGKRVGIIGLGRIGLAVAERAEAFDCPISYFSRSKKPNTNYTYYGSVVELASNSDILVVACPLTPETTHIINREVIDALGPKGVLINIGRGPHVDEPELVSALVEGRLGGAGLDVFEREPEVPEKLFGLENVVLLPHVGSGTVETRKVMADLVVGNLEAHFSGKPLLTPVV

SEQ ID NO: 21 - protein, 332 aa, synthetic construct
MKIIAYAVRDDERPFFDTWMKENPDVEVKLVPELLTEDNVDLAKGFDGADVAQQKDYTAEVLNKLADEGVKNISLRNVGVDNLDVPTVKARGLNISNVPAYSPNAIAELSVTQLMQLLRQTPLFNKKLAKQDFRWAPDIAKELNTMTVGVIGTGRIGRAAIDIFKGFGAKVIGYDVYRNAELEKEGMYVDTLDELYAQADVITLHVPALKDNYHMLNADAFSKMKDGAYILNFARGTLIDSEDLIKALDSGKVAGAALDTYEYETKIFNKDLEGQTIDDKVFMNLFNRDNVLITPHTAFYTETAVHNMVHVSMNSNKQFIETGKADTQVKFD

SEQ ID NO: 22 - protein, 689 aa, synthetic construct
MFIETNVAKPASTKAMNAGSAKAAPVEPFATYAHSQATKTVSIDGHTMKVGDVVAVARHGAKVELAASVAGPVRASVDFKESKKHTSIYGVTTGFGGSADTRTSDTEALQISLLEHQLCGFLPTDATYEGMLLAAMPIPIVRGAMAVRVNSCVRGHSGVRLEVLQSFADFINRGLVPCVPLRGTISASGDLSPLSYIAGAICGHPDVKVFDTAASPPTVLTSPEAIAKYGLKTVKLASKEGLGLVNGTAVSAAAGALALYDAECLAIMSQTNTVLTVEALDGHVGSFAPFIQEIRPHAGQIEAARNIRHMLGGSKLAVHEESELLADQDAGILRQDRYALRTSAQWIGPQLEALGLARQQIETELNSTTDNPLIDVEGGMFHHGGNFQAMAVTSAMDSARIVLQNLGKLSFAQVTELINCEMNHGLPSNLAGSEPSTNYHCKGLDIHCGAYCAELGFLANPMSNHVQSTEMHNQSVNSMAFASARRTMEANEVLSLLLGSQMYCATQALDLRVMEVKFKMAIVKLLNETLTKHFAAFLTPEQLAKLNTHAAITLYKRLNQTPSWDSAPRFEDAAKHLVGVIMDALMVNDDITDLTNLPKWKKEFAKEAGNLYRSILVATTADGRNDLEPAEYLGQTRAVYEAVRSELGVKVRRGDVAEGKSGKSIGSSVAKIVEAMRDGRLMGAVGKMF

SEQ ID NO: 23 - protein, 689 aa, synthetic construct; identical to SEQ ID NO: 22

SEQ ID NO: 24 - DNA, 6175 bp, synthetic construct
  source 1..6175: mol_type = other DNA
gaattcgtcacgatggttttcccaactcagtcaggattaaactgtgggtcagcgaaacgtttcgctgatggagaaaaaaatgaaaaaaggcaccgttcttaattctgatatttcatcggtgatctcccgtctgggacataccgatacgctggtggtgtgtgatgctggtttacccatccccaaaagtacaacgcgtatcgatatggcattaacccagggtgtaccttcttttatgcaggtgctgggcgtcgtcacaaatgaaatgcaggtcgaggcggccattatcgcggaagagatcaaacaccataatccgcaactccacgaaacgttgctcactcaccttgagcagctgcaaaaacaccagggaaataccattgaaattcgttacaccacgcatgaacaattcaaacaacaaaccgcagaaagtcaggcggtaattcgcagcggagaatgttctccgtatgcgaatatcattctctgtgctggcgtgacgttctgaggccgtcgcggccgccccgggatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaactgcagaagggcccatggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctattttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgtacagcgttttcagagtgaaagccgcgtgttattgcgtcaggcgaatgacaatcgccagtaaaagcttccccacgtgactagttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgatttacgctgtcttttgatcaaattattaccatcggttgttttcagaagcatgaacattgctgctgaagcaacagtaataatgcccattgtgataaacgtatagtggaattgttcgacagtcgttgtgccttccattccttcataaacgcgaaggacggccgcacttacagcaacgcctaaactaatcgacagttgctgcgtgaccgccagaacactgttaccgctgctggcattgtcatcggtcagatcggcaagtgtgatggtattcatcgcggtaaactgcgtcgacatagccatccctaatataaacaacggcaagatcagcatccatatagccattgctggtgattgcaaagagaactgagcgatcattagcccaataatcaccgtgatccccactaacgtatggcgatagcccagacgacgtaagacttgggtaaccatcgattttgcaataatggaacctaacgctgtcggtgccatcatacagccagcaatagaattcatcgtcgaacggcaggcgtgcaaacttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaatgggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagaactcggtacgcgcggatcttccagagatt

SEQ ID NO: 25 - DNA, 53 bp, synthetic construct
  source 1..53: mol_type = other DNA
aataaggagatataccatgggcatgaacaccattaatgaatacttgagtttag

SEQ ID NO: 26 - DNA, 49 bp, synthetic construct
  source 1..49: mol_type = other DNA
catggtatatctccttttaattgttaatcaaatgatccttaaccttttg

SEQ ID NO: 27 - DNA, 37 bp, synthetic construct
  source 1..37: mol_type = other DNA
taaaaggagatataccatgaaaccagaagatttccgc

SEQ ID NO: 28 - DNA, 44 bp, synthetic construct
  source 1..44: mol_type = other DNA
cattatgcggccgcaagcttttaaatcgcagcttccatttccag

SEQ ID NO: 29 - DNA, 55 bp, synthetic construct
  source 1..55: mol_type = other DNA
aagtataagaaggagatatacatatggaagctattggtgttttgatgatgtgtcc

SEQ ID NO: 30 - DNA, 48 bp, synthetic construct
  source 1..48: mol_type = other DNA
catggtatatctccttttaaacaacaggagttaacaatggtttaccag

SEQ ID NO: 31 - DNA, 53 bp, synthetic construct
  source 1..53: mol_type = other DNA
taaaaggagatataccatgaaaattgaagttaaagattcaactatgattaagc

SEQ ID NO: 32 - DNA, 59 bp, synthetic construct
  source 1..59: mol_type = other DNA
gtggcagcagcctaggttaattaaatttcataaaacaatttttcaaatctttccatatg

SEQ ID NO: 33 - DNA, 53 bp, synthetic construct
  source 1..53: mol_type = other DNA
aaggagatataccatgggcatgaattatcagaacgacgatttacgcatcaaag

SEQ ID NO: 34 - DNA, 40 bp, synthetic construct
  source 1..40: mol_type = other DNA
cattatgcggccgcaagcttttacccgcgacgcgctttta

SEQ ID NO: 35 - DNA, 22 bp, synthetic construct
  source 1..22: mol_type = other DNA
aagcttgcggccgcataatgct

SEQ ID NO: 36 - DNA, 32 bp, synthetic construct
  source 1..32: mol_type = other DNA
gcccatggtatatctccttattaaagttaaac

SEQ ID NO: 37 - DNA, 20 bp, synthetic construct
  source 1..20: mol_type = other DNA
ttaacctaggctgctgccac

SEQ ID NO: 38 - DNA, 30 bp, synthetic construct
  source 1..30: mol_type = other DNA
catatgtatatctccttcttatacttaact

SEQ ID NO: 39 - DNA, 20 bp, synthetic construct
  source 1..20: mol_type = other DNA
aagcttgcggccgcataatg

SEQ ID NO: 40 - DNA, 35 bp, synthetic construct
  source 1..35: mol_type = other DNA
gcccatggtatatctccttcttaaagttaaacaaa

SEQ ID NO: 41 - DNA, 47 bp, synthetic construct
  source 1..47: mol_type = other DNA
taaggagatataccatgggctttattgaaaccaacgtggcaaaaccg

SEQ ID NO: 42 - DNA, 46 bp, synthetic construct
  source 1..46: mol_type = other DNA
cattatgcggccgcaagcttttaaaacattttacccactgcaccca

SEQ ID NO: 43 - DNA, 49 bp, synthetic construct
  source 1..49: mol_type = other DNA
aagtataagaaggagatatacatatgaacatctctcgtcgtaaactgct

SEQ ID NO: 44 - DNA, 49 bp, synthetic construct
  source 1..49: mol_type = other DNA
gtggcagcagcctaggttaattattttttgaaacgatccaggctgaacg

SEQ ID NO: 45 - DNA, 43 bp, synthetic construct
  source 1..43: mol_type = other DNA
aagtataagaaggagatatacatatggcgccgcaggaacaggc

SEQ ID NO: 46 - DNA, 45 bp, synthetic construct
  source 1..45: mol_type = other DNA
gtggcagcagcctaggttaattacaggccgttcgccagtttcgca

SEQ ID NO: 47 - DNA, 57 bp, synthetic construct
  source 1..57: mol_type = other DNA
taaaagcgcgtcgcgggtaaaaggagatataccatgaccgttttcaaacacatcgcg

SEQ ID NO: 48 - DNA, 43 bp, synthetic construct
  source 1..43: mol_type = other DNA
cattatgcggccgcaagcttttacgggtggatatcgtgatcgg

SEQ ID NO: 49 - DNA, 22 bp, synthetic construct
  source 1..22: mol_type = other DNA
aagcttgcggccgcataatgct

SEQ ID NO: 50 - DNA, 22 bp, synthetic construct
  source 1..22: mol_type = other DNA
ttacccgcgacgcgcttttact

SEQ ID NO: 51 - DNA, 24 bp, synthetic construct
  source 1..24: mol_type = other DNA
ctcataaagctaacccgccgtttt

SEQ ID NO: 52 - DNA, 24 bp, synthetic construct
  source 1..24: mol_type = other DNA
cgttgtcaccagaaaagtgtgacg

SEQ ID NO: 53 - DNA, 53 bp, synthetic construct
  source 1..53: mol_type = other DNA
cacacttttctggtgacaacgtcatttaataatctccagtaaagcctgcacag

SEQ ID NO: 54 - DNA, 34 bp, synthetic construct
  source 1..34: mol_type = other DNA
tactttgttatcgcgatgaatataaactggcact

SEQ ID NO: 55 - DNA, 45 bp, synthetic construct
  source 1..45: mol_type = other DNA
ttgaaatcttcgatgagaaagttttagagctagaaatagcaagtt

SEQ ID NO: 56 - DNA, 45 bp, synthetic construct
  source 1..45: mol_type = other DNA
tttctcatcgaagatttcaaactagtattatacctaggactgagc

SEQ ID NO: 57 - DNA, 27 bp, synthetic construct
  source 1..27: mol_type = other DNA
gtgcagcgttcagaaataagaaaaccc

SEQ ID NO: 58 - DNA, 23 bp, synthetic construct
  source 1..23: mol_type = other DNA
ccaaatggcaaagcccagcatat

(19) United States
(12) Patent Application Publication
(10) Pub. No.: US 2024/0084338 A1
(43) Pub. Date: Mar. 14, 2024

(54) Recombinant Escherichia coli for producing rosmarinic acid and its application thereof

(71) Applicant: Jiangnan University, Wuxi (CN)

(72) Inventors: Jingwen ZHOU, Wuxi (CN); Jian Chen, Wuxi (CN); Lian Wang, Wuxi (CN); Weizhu Zeng, Wuxi (CN); Shiqin Yu, Wuxi (CN)

(21) Appl. No.: 18/515,572

(22) Filed: Nov. 21, 2023

(30) Foreign Application Priority Data
Nov. 22, 2022 (CN) 2022114686177

(51) Int. Cl. C12P 7/62; C12N 9/00; C12N 9/04; C12N 9/06; C12N 9/10; C12N 9/88; C12N 15/52; C12N 15/70

(52) U.S. Cl. CPC C12P 7/62; C12N 9/0006; C12N 9/0028; C12N 9/1029; C12N 9/1085; C12N 9/88; C12N 9/93; C12N 15/52; C12N 15/70; C12Y 101/01237; C12Y 105/01; C12Y 203/0114; C12Y 205/01054; C12Y 403/01017; C12Y 403/01023; C12Y 602/01012

(57) ABSTRACT

The present disclosure discloses a recombinant Escherichia coli for producing rosmarinic acid and application thereof, belonging to the technical fields of genetic engineering and bioengineering. In the present disclosure, FjTA derived from Flavobacterium johnsoniae, endogenous hpaBC derived from E. coli, CbRAS derived from Coleus blumei, HPPR derived from Coleus scutellarioides, and Pc4CL1 derived from Petroselinum crispum are heterologously expressed in E. coli, realizing synthesis of rosmarinic acid. TcTAL derived from Trichosporon cutaneum and tyrC for removing feedback inhibition are introduced, further increasing synthesis throughput of caffeic acid, and PmLAAD derived from Proteus myxofaciens is heterologously expressed, realizing redistribution of L-DOPA. An endogenous gene menl is knocked out, improving the content and stability of a rosmarinic acid precursor. The recombinant strain constructed in the present disclosure can produce rosmarinic acid by fermentation at a yield of up to 511.2 mg/L, providing a new method for industrial production of rosmarinic acid.

The instant application contains a Sequence Listing in XML format as a file named “YGHY-2023-58-SEQ.xml”, created on Oct. 21, 2023, of 78 kB in size, and which is hereby incorporated by reference in its entirety.

### TECHNICAL FIELD

The present disclosure relates to a recombinant Escherichia coli for producing rosmarinic acid and application thereof, belonging to the technical fields of genetic engineering and bioengineering.

### BACKGROUND

Rosmarinic acid is a naturally occurring phenolic acid compound, and named after its earliest discovery in rosemary. Structurally, rosmarinic acid is an ester formed by condensation of caffeic acid and salvianic acid A. Rosmarinic acid shows great application potential in industries such as health-care products and medicine. Like most phenolic acid compounds, rosmarinic acid has good antioxidant activity. In addition, rosmarinic acid may also be used as a precursor of the important compound salvianolic acid B in Salvia miltiorrhiza. The synthesis of rosmarinic acid has received more and more attention from researchers.

Currently, the production of rosmarinic acid mainly relies on plant extraction, but the production efficiency is affected by factors such as season and low plant tissue content. Some researchers use plant cell suspensions to synthesize rosmarinic acid, but the production cycle of this method is relatively long and the production scale is difficult to expand. The traditional chemical synthesis of rosmarinic acid involves multi-step reactions, and some reagents are expensive, which makes the traditional chemical synthesis not suitable for large-scale production.

### SUMMARY

The present disclosure provides a recombinant E. coli for synthesizing rosmarinic acid, which, on the basis of an original strain, expresses tyrosine ammonia-lyase FjTAL derived from Flavobacterium johnsoniae, 4-hydroxyphenylacetate-3-monooxygenase hpaBC derived from E. coli, a 3-deoxy-D-arabino-heptulosonate-7-phosphate synthase mutant aroG^{fbr}, rosmarinic acid synthase CbRAS derived from Coleus blumei, hydroxyphenylpyruvate reductase HPPR derived from Coleus scutellarioides, and 4-coumarate:coenzyme A ligase 4CL1.

In one embodiment, the 4-coumarate:coenzyme A ligase is 4-coumarate:coenzyme A ligase Pc4CL1 derived from Petroselinum crispum, having an amino acid sequence shown in a UniProt accession number: P14912.1 (SEQ ID NO.12).

In one embodiment, the 4-coumarate:coenzyme A ligase is 4-coumarate:coenzyme A ligase At4CL1 derived from Arabidopsis thaliana, having an amino acid sequence shown in an NCBI accession number: NP_175579.1 (SEQ ID NO.13).

In one embodiment, the recombinant E. coli expresses the tyrosine ammonia-lyase FjTAL derived from F. johnsoniae, the 4-hydroxyphenylacetate-3-monooxygenase hpaBC derived from E. coli, the 3-deoxy-D-arabino-heptulosonate-7-phosphate synthase mutant aroG^{fbr}, the rosmarinic acid synthase CbRAS derived from C. blumei, the hydroxyphenylpyruvate reductase HPPR derived from C. scutellarioides, and the 4-coumarate:coenzyme A ligase Pc4CL1 derived from P. crispum.

In one embodiment, the recombinant E. coli expresses the tyrosine ammonia-lyase FjTAL derived from F. johnsoniae, the 4-hydroxyphenylacetate-3-monooxygenase hpaBC derived from E. coli, the 3-deoxy-D-arabino-heptulosonate-7-phosphate synthase mutant aroG^{fbr}, the rosmarinic acid synthase CbRAS derived from C. blumei, the hydroxyphenylpyruvate reductase HPPR derived from C. scutellarioides, chorismate mutase tyrC derived from Zymomonas mobilis, and the 4-coumarate:coenzyme A ligase At4CL1 derived from A. thaliana.

In one embodiment, an amino acid sequence of the aroG mutant aroG^{fbr} is shown in a Genbank accession number: AXN70009.1 (SEQ ID NO.14), and the mutant is a mutant of the 3-deoxy-D-arabino-heptulosonate-7-phosphate synthase with phenylalanine feedback inhibition removed.

In one embodiment, the chorismate mutase tyrC derived from Z. mobilis has an amino acid sequence shown in a Genbank accession number: AAA27684.1 (SEQ ID NO.15).

In one embodiment, a Genbank accession number of the tyrosine ammonia-lyase is WP_012023194.1 (SEQ ID NO.16); Genbank accession numbers of the hpaBC are WP_000801472.1 (SEQ ID NO.17) and WP_001175451.1 (SEQ ID NO.18); a UniProt accession number of the rosmarinic acid synthase is A0PDV5.1 (SEQ ID NO.19); a Genbank accession number of the hydroxyphenylpyruvate reductase is Q65CJ7.2 (SEQ ID NO.20); and a UniProt accession number of the 4-coumarate:coenzyme A ligase Pc4CL1 is P14912.1 (SEQ ID NO.12).

In one embodiment, the hydroxyphenylpyruvate reductase gene HPPR derived from C. scutellarioides is replaced with a lactate dehydrogenase gene ldh (Genbank accession number: WP_003640741.1, SEQ ID NO.21) derived from Lactiplantibacillus plantarum.

In one embodiment, the recombinant E. coli expresses tyrosine ammonia-lyase TcTAL (Genbank accession number: AKE50834.1, SEQ ID NO.22) derived from Trichosporon cutaneum.

In one embodiment, the recombinant E. coli expresses tyrosine ammonia-lyase TcTAL (Genbank accession number: AKE50834.1, SEQ ID NO.22) derived from T. cutaneum, and also expresses L-amino acid deaminase PmLAAD (Genbank accession number: AXQ04983.1, SEQ ID NO.23) derived from Proteus myxofaciens.

In one embodiment, the recombinant E. coli has an endogenous thioesterase menl knocked out, which improves the content and stability of a rosmarinic acid precursor.

In one embodiment, the recombinant E. coli uses E. coli BL21(DE3) ΔtyrRΔcrrΔptsGΔpheA as an original strain. The E. coli BL21(DE3) ΔtyrRΔcrrΔptsGΔpheA is disclosed in paper “Enhancing caffeic acid production in Escherichia coli by engineering the biosynthesis pathway and transporter”.

In one embodiment, the nucleotide sequences of the genes FjTAL, hpaBC, CbRAS, HPPR and Pc4CL1 are shown in SEQ ID NO.1, SEQ ID NO.2, SEQ ID NO.3, SEQ ID NO.4 and SEQ ID NO.5.

In one embodiment, a sequence of the aroG^{fbr} gene is shown in SEQ ID NO.6.

In one embodiment, a nucleotide sequence of the TcTAL gene is shown in SEQ ID NO.7.

In one embodiment, a sequence of the PmLAAD gene is shown in SEQ ID NO.8.

In one embodiment, a sequence of the At4CL1 gene is shown in SEQ ID NO.9.

In one embodiment, a sequence of the tyrC gene is shown in SEQ ID NO.10.

In one embodiment, a sequence of the ldh gene is shown in SEQ ID NO.11.

In one embodiment, pETDuet-1 is used as an expression vector to express the aroG^{fbr} gene, and the HPPR gene and the CbRAS gene.

In one embodiment, pACYCDuet-1 is used as an expression vector to express the FjTAL, hpaBC and Pc4CL1 genes.

In one embodiment, pACYCDuet-1 is used as an expression vector to express the FjTAL, hpaBC and At4CL1 genes.

In one embodiment, pCDFDuet-1 is used as an expression vector to express the TcTAL gene and the PmLAAD gene.

The present disclosure further provides application of the recombinant E. coli in production of rosmarinic acid or derivatives thereof by fermentation.

The present disclosure provides a method for producing rosmarinic acid, which uses the recombinant E. coli to produce rosmarinic acid by fermentation.

In one embodiment, the recombinant E. coli is inoculated into a fermentation system and cultured at 37° C. for 3-4 h, IPTG with a final concentration of 0.1-0.2 mM is added, and induction and fermentation are carried out at 30° C. at 200-220 r/min for 24-72 h to synthesize rosmarinic acid.

In one embodiment, the fermentation system contains: glucose, glycerol, (NH₄)₂SO₄, K₂HPO₄·3H₂O, KH₂PO₄, MgSO₄·7H₂O, sodium citrate, vitamin B1, yeast extract, vitamin C and betaine.

The present disclosure provides application of the recombinant E. coli in production of rosmarinic acid and derivatives thereof.

In one embodiment, the derivatives include, but not limited to, methyl rosmarinate, ethyl rosmarinate and salvianolic acid B.

### Beneficial Effects

In the present disclosure, the E. coli BL21(DE3) ΔtyrRΔcrrΔptsGΔpheA is used as the host to express the tyrosine ammonia-lyase FjTAL derived from F. johnsoniae, the endogenous hpaBC of the E. coli BL21(DE3), the rosmarinic acid synthase gene CbRAS derived from C. blumei, the Pc4CL1 derived from P. crispum, and the hydroxyphenylpyruvate reductase HPPR derived from C. scutellarioides or the lactate dehydrogenase gene ldh derived from the L. plantarum, so as to synthesize rosmarinic acid. Then, tyrosine ammonia-lyase TcTAL derived from T. cutaneum and tyrC for removing feedback inhibition are introduced to reduce the accumulation of the product intermediate L-DOPA of caffeic acid synthesis, thereby further increasing synthesis throughput of caffeic acid, and the L-amino acid deaminase PmLAAD realizes transformation of the L-DOPA to 3,4-dihydroxyphenylpyruvic acid. Then, the encoding endogenous thioesterase gene menl is knocked out to improve the intracellular stability of caffeoyl coenzyme A, thereby improving the content and stability of the rosmarinic acid precursor. Finally, different hydroxyphenylpyruvate reductases are compared to further increase the accumulation of rosmarinic acid. Under shake flask fermentation conditions, the accumulation of rosmarinic acid reaches 511.2 mg/L. In the present disclosure, a plant heterologous biosynthesis pathway is reconstructed in E. coli, thereby providing a new idea for efficient production of rosmarinic acid and biosynthesis of the derivative salvianolic acid B.

### BRIEF DESCRIPTION OF FIGURES

FIG. 1 is a schematic diagram showing metabolism of heterologous biosynthesis of rosmarinic acid in E. coli;

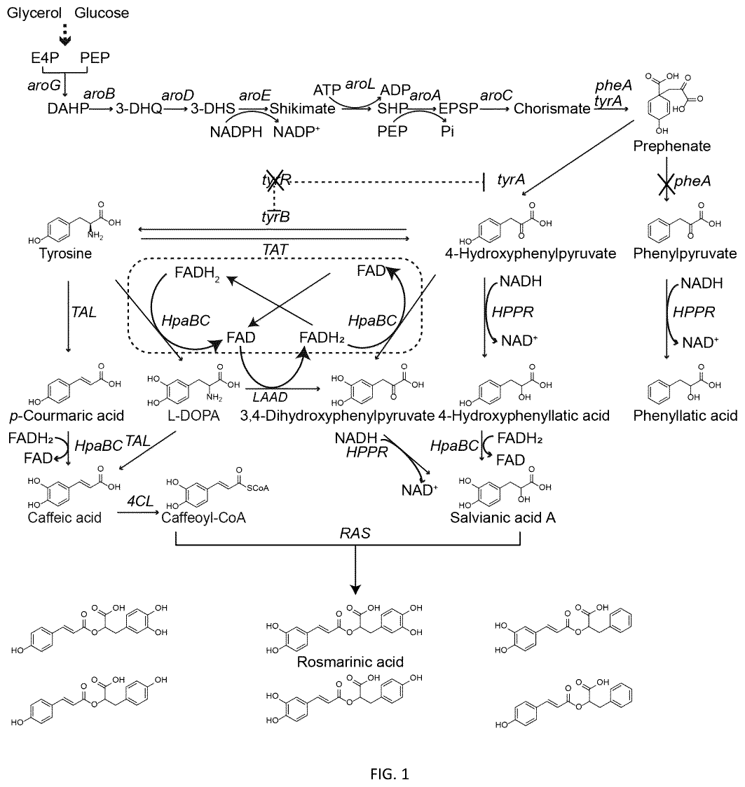

FIG. 2 is shows a rosmarinic acid chromatogram of E. coli RA15 cultured in a fermentation medium and a rosmarinic acid standard chromatogram;

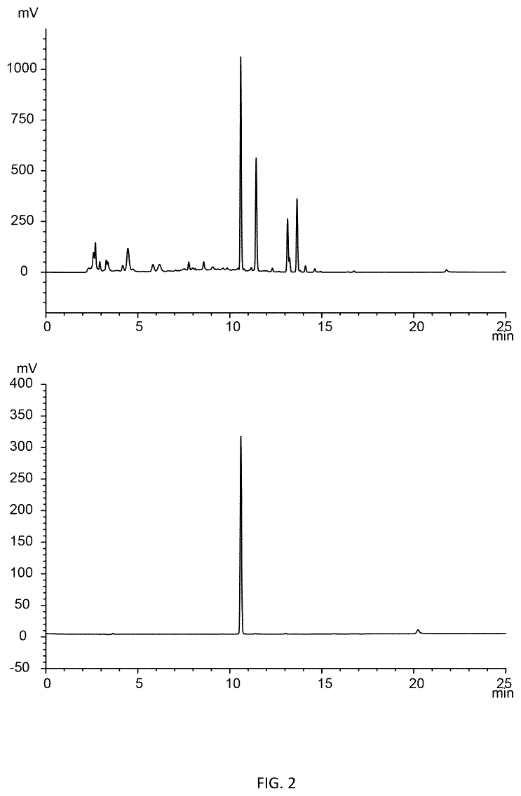

FIG. 3 is a titer diagram of rosmarinic acid of E. coli cultured in a fermentation medium.

### DETAILED DESCRIPTION

(I) Culture media

Seed medium (LB): peptone 10 g/L, yeast extract 5 g/L, and sodium chloride 5 g/L; and 2% (mass fraction) agar powder added to the solid medium.

Fermentation medium: glucose 25 g/L, glycerol 10 g/L, (NH₄)₂SO₄ 7.5 g/L, K₂HPO₄·3H₂O 3 g/L, KH₂PO₄ 2 g/L, MgSO₄·7H₂O 2 g/L, sodium citrate 1 g/L, vitamin B1 0.1 g/L, yeast extract 7 g/L and betaine 5 g/L. 250 g/L glucose is sterilized separately and mixed evenly before inoculation.
(II) PCR reaction system and amplification conditions: forward primer (10 μM) 1 μL, reverse primer (10 μM) 1 μL, template DNA 10-50 ng, 2× Phanta Max Master Mix 25 μL, and double distilled water added to 50 μl. Amplification conditions: pre-denaturation at 95° C. for 3 min; 30 cycles (95° C. for 15 s, 55° C. for 15 s, and 72° C. for 15 s), and extension at 72° C. for 5 min.
(III) Preparation of E. coli competent cells: A glycerol tube of E. coli JM109 was streaked on a corresponding LB plate, and cultured at 37° C. overnight (for about 12 h). After 12 h, monoclonal cells were picked and inoculated into a 50 mL shake flask containing 5 mL of LB medium, and then cultured at 37° C. at 220 rpm until OD 600=0.6. The bacterial solution was transferred to a 50 mL centrifuge tube, and placed on ice for about 15 min. The centrifuge tube was centrifuged at 4000 rpm at 4° C. for 5 min, and the supernatant was removed. 5 mL of solution A was added for resuspension. The centrifuge tube was centrifuged at 4000 r/min at 4° C. for 5 min, and the supernatant was removed. 5 mL of solution B was added to resuspend the cells, divided according to 100 μL/package, and stored at −80° C.
(IV) Transformation of E. coli: The E. coli competent cells were thawed on ice. 10 μl of recombinant product (plasmid 50 ng) was added into 100 μl of competent cells, evenly mixed by flicking, and allowed to stand on ice for 30 min. The competent cells were subjected to heat shock in a 42° C. water bath for 45 s, and allowed to stand on ice for 2 min. 1 mL of LB medium was added, and the cells were shaken at 37° C. at 220 rpm for 60 min. The bacterial solution was centrifuged at 4500 rpm for 2 min, and 900 μl of supernatant was removed. The cells were resuspended with the remaining culture medium, and then the bacterial solution was coated on a resistant plate.
(V) Determination of rosmarinic acid by HPLC: After the fermentation was completed, 500 μl of fermentation broth was added to the same volume of methanol, and then shaken vigorously and mixed uniformly. The mixture was centrifuged at 14000 r/min for 10 min. The supernatant was filtered through a 0.22 μm organic phase filter membrane, and a Shimadzu LC-20A high-performance liquid chromatograph was used to detect the product. A Thermo Fisher C18 column (4.6 mm×250 mm, 5 μm) was used for chromatographic separation. The temperature of the column oven was set to 40° C. The injection volume was 10 μL. The mobile phases were: phase A: ultrapure water (with 0.1% trifluoroacetic acid), and phase B: acetonitrile (with 0.1% trifluoroacetic acid). The total flow rate was 1 mL/min. The type of elution was gradient elution: 0-10 min, phase B: 10-60%; 10-20 min, phase B: 60-80%; 20-22 min, phase B: 80-10%; 22-25 min, phase B: 10%. The detector wavelength was 323 nm.
(VI) The information of strains is shown in Table 1:

TABLE 1
Strains and genotypes involved in the present disclosure

| Strain Name | Genotype |
|---|---|
| E. coli BL21(BE3) | E. coli BL21 (DE3) with tyrR, crr, ptsG and pheA knocked out |
| ΔtyrRΔcrrΔptsGΔpheA | |
| E. coli BL21 (DE3) | E. coli BL21 (DE3) with tyrR, crr, ptsG, pheA and menI knocked out |
| ΔtyrRΔcrrΔptsGΔpheAΔmenI | |
| RA04 | E. coli BL21 (DE3) ΔtyrRΔcrrΔptsGΔpheA containing pETDuet- |
| | aroG^{fbr} -HPPR-CbRAS and pACYCDuet-FjTAL-RBS-HpaBC-Pc4CL1 |
| RA05 | E. coli BL21 (DE3) ΔtyrRΔcrrΔptsGΔpheA containing pETDuet- |
| | aroG^{fbr} -HPPR-CbRAS, pACYCDuet-FjTAL-RBS-HpaBC-Pc4CL1 and |
| | pCDF-TcTAL |
| RA06 | E. coli BL21 (DE3) ΔtyrRΔcrrΔptsGΔpheA containing pETDuet- |
| | aroG^{fbr} -HPPR-CbRAS, pACYCDuet-FjTAL-RBS-HpaBC-Pc4CL1 and |
| | pCDF-TcTAL-PmLAAD |
| RA08 | E. coli BL21 (DE3) ΔtyrRΔcrrΔptsGΔpheA containing pETDuet- |
| | aroG^{fbr} -tyrC-HPPR-CbRAS, pACYCDuet-FjTAL-RBS-HpaBC-At4CL1 |
| | and pCDF-TcTAL |
| RA09 | E. coli BL21 (DE3) ΔtyrRΔcrrΔptsGΔpheA containing pETDuet- |
| | aroG^{fbr} -tyrC-HPPR-CbRAS, pACYCDuet-FjTAL-RBS-HpaBC-At4CL1 |
| | and pCDF-TcTAL-PmLAAD |
| RA10 | E. coli BL21 (DE3) ΔtyrRΔcrrΔptsGΔpheAΔmenI containing |
| | pETDuet- aroG^{fbr} -tyrC-HPPR-CbRAS, pACYCDuet-FjTAL-RBS-HpaBC- |
| | At4CL1 and pCDF-TcTAL-PmLAAD |
| RA15 | E. coli BL21 (DE3) ΔtyrRΔcrrΔptsGΔpheAΔmenI containing |
| | pETDuet- aroG^{fbr} -tyrC-Idh-CbRAS, pACYCDuet-FjTAL-RBS-HpaBC- |
| | At4CL1 and pCDF-TcTAL-PmLAAD |

### Example 1: Construction of Recombinant E. coli for Synthesizing Rosmarinic Acid

E. coli BL21(DE3) ΔtyrRΔcrrΔptsGΔpheA (disclosed in paper “Enhancing caffeic acid production in Escherichia coli by engineering the biosynthesis pathway and transporter”) was used as an original strain for synthesizing rosmarinic acid. First, in order to construct the synthesis pathway of the precursor caffeic acid for synthesizing rosmarinic acid, using the synthesized FjTAL sequence (the nucleotide sequence shown in SEQ ID NO.1) as a template, PCR amplification was carried out on the FjTAL fragment by using a primer pair F1/R1. Using the E. coli BL21(DE3) genome as a template, PCR amplification was carried out on the hpaBC fragment (the nucleotide sequence shown in SEQ ID NO.2) by using a primer pair F2/R2. Using a pACYCDuet-1 vector as a template, amplification was carried out by using primers FP1/RP1, and the product was purified to obtain a pACYCDuet-1 skeleton fragment. The FjTAL fragment, the hpaBC fragment and the vector pACYCDuet-1 skeleton were recombined by Gibson assembly to obtain a recombinant vector, and the recombinant vector was transformed into E. coli JM109. Plasmid extraction was carried out, and sequencing was carried out for verification, thereby obtaining a correct recombinant vector pACYCDuet-FjTAL-RBS-HpaBC (RBS sequence: AAGGAGATATACC). Using the synthesized Pc4CL1 (the nucleotide sequence shown in SEQ ID NO.5) as a template, amplification was carried out by using a primer pair F3/R3, and the product was purified to obtain a Pc4CL1 fragment. Using pACYCDuet-FjTAL-RBS-HpaBC as a template, amplification was carried out by using a primer pair M2-F/M2-R, and the product was purified to obtain a pACYCDuet-FjTAL-RBS-HpaBC skeleton fragment. The Pc4CL1 fragment and the vector pACYCDuet-FjTAL-RBS-HpaBC skeleton were recombined by Gibson assembly to obtain a recombinant vector, and the recombinant vector was transformed into E. coli JM109. Plasmid extraction was carried out, and sequencing was carried out for verification, thereby obtaining a correct recombinant vector pACYCDuet-FjTAL-RBS-HpaBC-Pc4CL1.

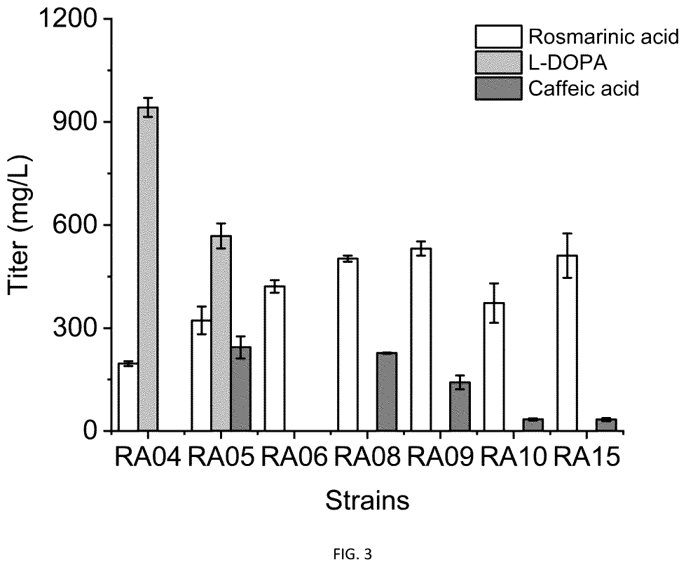

In order to construct the synthesis pathway of rosmarinic acid, using the synthesized HPPR sequence (the nucleotide sequence shown in SEQ ID NO.4) as a template, amplification was carried out by using the primer pair F3/R3, and the product was purified to obtain a HPPR fragment. Using the synthesized CbRAS (the nucleotide sequence shown in SEQ ID NO.3) as a template, amplification was carried out by using a primer pair F4/R4, and the product was purified to obtain a CbRAS fragment. Using a pETDuet-1 vector as a template, amplification was carried out by using the primer M2-F/M2-R, and the product was purified to obtain a pETDuet-1 vector skeleton. The HPPR fragment, the CbRAS fragment and the vector pETDuet-1 skeleton were recombined by Gibson assembly to obtain a recombinant vector, and the recombinant vector was transformed into E. coli JM109. Plasmid extraction was carried out, and sequencing was carried out for verification, thereby obtaining the correct recombinant vector pETDuet-HPPR-CbRAS. Using pMD-tyrA^{fbr}-aroG^{fbr} (disclosed in paper “Fermentation and Metabolic Pathway Optimization to De Novo Synthesize (2S)-Naringenin in Escherichia coli”, shown in SEQ ID NO.24) as a template, amplification was carried out on an aroG^{fbr} fragment (the nucleotide sequence shown in SEQ ID NO.6) by using a primer pair F5/R5, and the product was purified. Using the recombinant vector pETDuet-HPPR-CbRAS as a template, amplification was carried out by using a primer pair FP2/RP2, and the product was purified to obtain a pETDuet-HPPR-CbRAS skeleton. The fragment aroG^{fbr} and the vector pETDuet-HPPR-CbRAS skeleton were recombined by Gibson assembly to obtain a recombinant vector, and the recombinant vector was transformed into E. coli JM109. Plasmid extraction was carried out, and sequencing was carried out for verification, thereby obtaining a correct recombinant vector pETDuet-aroG fb r-HPPR-CbRAS. The recombinant vectors pACYCDuet-FjTAL-RBS-HpaBC-Pc4CL1 and pETDuet-aroG^{fbr}-HPPR-CbRAS were transformed into E. coli BL21(DE3) ΔtyrRΔcrrΔptsGΔpheA to obtain an engineering strain RA04. The engineering strain was cultured in the seed medium at 37° C. at 220 r/min for 12 h to obtain a seed solution, and the seed solution was inoculated into a fermentation medium containing 100 μg/mL (final concentration) ampicillin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 24 h to synthesize rosmarinic acid. As shown in FIG. 3, in the fermentation broth of RA04, the yield of rosmarinic acid was 195.9 mg/L, the yield of L-DOPA was 942.2 mg/L, and no accumulation of caffeic acid was detected.

All primer sequences are listed in Table 2.

TABLE 2
Primer sequences

| Primer | | SEQ ID |
|---|---|---|
| Name | Primer Sequence | NUNBER |
| F1 | AATAAGGAGATATACCATGGGCATGAACACCATTAATGAATACTTGAGTTT | SEQ ID NO. 25 |
| | AG | |
| R1 | CATGGTATATCTCCTTTTAATTGTTAATCAAATGATCCTTAACCTTTTG | SEQ ID NO. 26 |
| F2 | TAAAAGGAGATATACCATGAAACCAGAAGATTTCCGC | SEQ ID NO. 27 |
| R2 | CATTATGCGGCCGCAAGCTTTTAAATCGCAGCTTCCATTTCCAG | SEQ ID NO. 28 |
| F3 | AAGTATAAGAAGGAGATATACATATGGAAGCTATTGGTGTTTTGATGATGT | SEQ ID NO. 29 |
| | GTCC | |
| R3 | CATGGTATATCTCCTTTTAAACAACAGGAGTTAACAATGGTTTACCAG | SEQ ID NO. 30 |
| F4 | TAAAAGGAGATATACCATGAAAATTGAAGTTAAAGATTCAACTATGATTAA | SEQ ID NO. 31 |
| | GC | |
| R4 | GTGGCAGCAGCCTAGGTTAATTAAATTTCATAAAACAATTTTTCAAATCTTT | SEQ ID NO. 32 |
| | CCATATG | |
| F5 | AAGGAGATATACCATGGGCATGAATTATCAGAACGACGATTTACGCATCAA | SEQ ID NO. 33 |
| | AG | |
| R5 | CATTATGCGGCCGCAAGCTTTTACCCGCGACGCGCTTTTA | SEQ ID NO. 34 |
| FP1 | AAGCTTGCGGCCGCATAATGCT | SEQ ID NO. 35 |
| RP1 | GCCCATGGTATATCTCCTTATTAAAGTTAAAC | SEQ ID NO. 36 |
| M2-F | TTAACCTAGGCTGCTGCCAC | SEQ ID NO. 37 |
| M2-R | CATATGTATATCTCCTTCTTATACTTAACT | SEQ ID NO. 38 |
| FP2 | AAGCTTGCGGCCGCATAATG | SEQ ID NO. 39 |
| RP2 | GCCCATGGTATATCTCCTTCTTAAAGTTAAACAAA | SEQ ID NO. 40 |

### Example 2: Overexpression of TcTAL to Improve Supply of Caffeic Acid

In order to further increase the transformation of L-DOPA to caffeic acid, using the synthesized TcTAL (the nucleotide sequence shown in SEQ ID NO.7) as a template, amplification was carried out by using a primer pair F6/R6, and the product was purified. Using a pCDFDuet-1 vector as a template, amplification was carried out by using primers FP1/RP1, and the product was purified. The fragment TcTAL and the vector pCDFDuet-1 skeleton were recombined by Gibson assembly to obtain a recombinant vector, and the recombinant vector was transformed into E. coli JM109. Plasmid extraction was carried out, and sequencing was carried out for verification, thereby obtaining a correct recombinant vector pCDFDuet-TcTAL. The recombinant vector pCDFDuet-TcTAL was transformed into the strain RA04 constructed in Example 1 to obtain an engineering strain RA05. The engineering strain RA05 was cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 72 h to synthesize rosmarinic acid. As shown in FIG. 3, in the fermentation broth of RA05, the titer of rosmarinic acid was 338.0 mg/L, the yield of L-DOPA was 568.0 mg/L, and the titer of caffeic acid was 243.4 mg/L.

All primer sequences are listed in Table 3.

TABLE 3
Primer sequence

| Primer | | SEQ ID |
|---|---|---|
| Name | Primer Sequence | NUNBER |
| F6 | TAAGGAGATATACCATGGGCTTTATTGAAACCAACGTGGCAAAAC | SEQ ID NO. 41 |
| | CG | |
| R6 | CATTATGCGGCCGCAAGCTTTTAAAACATTTTACCCACTGCACCCA | SEQ ID NO. 42 |

### Example 3: Heterologous Expression of L-Tyrosine Deaminase to Promote Redistribution of L-DOPA

In order to further reduce the accumulation of intracellular L-DOPA and balance synthesis flux of the two rosmarinic acid precursors caffeic acid and L-DOPA, using the synthesized PmLAAD (the nucleotide sequence shown in SEQ ID NO.8) as a template, amplification was carried out by using a primer pair F7/R7, and the product was purified to obtain a PmLAAD fragment. Using the recombinant plasmid pCDFDuet-TcTAL as a template, amplification was carried out by using primers M2-F/M2-R, and the product was purified to obtain a vector pCDFDuet-TcTAL skeleton. The PmLAAD fragment and the vector pCDFDuet-TcTAL skeleton were recombined by Gibson assembly to obtain a recombinant plasmid, and the recombinant plasmid was transformed into E. coli JM109. Plasmid extraction was carried out, and sequencing was carried out for verification, thereby obtaining a correct recombinant plasmid pCDFDuet-TcTAL-PmLAAD. The recombinant vector pCDFDuet-TcTAL-PmLAAD was transformed into the strain RA04 constructed in Example 1 to obtain an engineering strain RA06. The engineering strain was cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 24 h to synthesize rosmarinic acid. As shown in FIG. 3, in the fermentation broth of RA06, the titer of rosmarinic acid was 421.5 mg/L, and no accumulation of L-DOPA or caffeic acid was detected.

All primer sequences are listed in Table 4.

TABLE 4
Primer sequence

| Primer | | SEQ ID |
|---|---|---|
| Name | Primer Sequence | NUMBER |
| F7 | AAGTATAAGAAGGAGATATACATATGAACATCTCTCGTCGTAAACTG | SEQ ID |
| | CT | NO. 43 |
| R7 | GTGGCAGCAGCCTAGGTTAATTATTTTTTGAAACGATCCAGGCTGAA | SEQ ID |
| | CG | NO. 44 |

### Example 4: Optimization of Selectivity of 4CL and Removal of Feedback Inhibition of Tyrosine

As a direct precursor for the synthesis of rosmarinic acid, the content of caffeoyl coenzyme A is directly affected by the enzyme activity and preference of 4-coumarate:coenzyme A. In order to increase the content of the caffeoyl coenzyme A, using the synthesized At4CL1 (the nucleotide sequence shown in SEQ ID NO.9) as a template, amplification was carried out by using a primer pair F8/R8, and the fragment was purified to obtain an At4CL1 fragment. The At4CL1 fragment and the vector pACYCDuet-FjTAL-RBS-HpaBC skeleton constructed in Example 1 were recombined by Gibson assembly to obtain a recombinant vector, and the recombinant vector was transformed into E. coli JM109. Plasmid extraction was carried out, and sequencing was carried out for verification, thereby obtaining a correct recombinant vector pACYCDuet-FjTAL-RBS-HpaBC-At4CL1.

Moreover, in order to further remove the feedback inhibition of tyrosine, using the synthesized tyrC (the nucleotide sequence shown in SEQ ID NO.10) as a template, amplification was carried out by using a primer pair F9/R9, and the fragment was purified to obtain a tyrC fragment. Using the recombinant vector pETDuet-aroG^{fbr}-HPPR-CbRAS constructed in Example 1 as a template, amplification was carried out by using a primer pair M1-F/aroG-R, and purification was carried out to obtain a pETDuet-aroG^{fbr}-HPPR-CbRAS skeleton. The tyrC fragment and the vector pETDuet-aroG^{fbr}-HPPR-CbRAS skeleton were recombined by Gibson assembly to obtain a recombinant vector, and the recombinant vector was transformed into E. coli JM109. Plasmid extraction was carried out, and sequencing was carried out for verification, thereby obtaining the correct recombinant vector pETDuet-aroG^{fbr}-tyrC-HPPR-CbRAS. The recombinant vectors pACYCDuet-FjTAL-RBS-HpaBC-At4CL1, and pCDFDuet-TcTAL and pETDuet-aroG^{fbr}-tyrC-HPPR-CbRAS constructed in Example 2 were transformed into E. coli BL21(DE3) ΔtyrRΔcrrΔptsGΔpheA to obtain an engineering strain RA08. The engineering strain RA08 was cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 72 h to synthesize rosmarinic acid. As shown in FIG. 3, in the fermentation broth of RA08, the yield of rosmarinic acid was 502.0 mg/L, no accumulation of L-DOPA was detected, and the yield of caffeic acid was 226.4 mg/L.

All primer sequences are listed in Table 5.

TABLE 5
Primer sequence

| Primer | | SEQ ID |
|---|---|---|
| Name | Primer Sequence | NUMBER |
| F8 | AAGTATAAGAAGGAGATATACATATGGCGCCGCAGGAACAGGC | SEQ ID NO. 45 |
| R8 | GTGGCAGCAGCCTAGGTTAATTACAGGCCGTTCGCCAGTTTCGCA | SEQ ID NO. 46 |
| F9 | TAAAAGCGCGTCGCGGGTAAAAGGAGATATACCATGACCGTTTTCAAA | SEQ ID NO. 47 |
| | CACATCGCG | |
| R9 | CATTATGCGGCCGCAAGCTTTTACGGGTGGATATCGTGATCGG | SEQ ID NO. 48 |
| M1-F | AAGCTTGCGGCCGCATAATGCT | SEQ ID NO. 49 |
| aroG-R | TTACCCGCGACGCGCTTTTACT | SEQ ID NO. 50 |

### Example 5: Heterologous Expression of L-Tyrosine Deaminase Based on Removal of Feedback Inhibition of Tyrosine and Optimization of Selectivity of 4CL

In Example 3 and Example 4, it was found that heterologous expression of L-tyrosine deaminase, removal of feedback inhibition of tyrosine and optimization of selectivity of 4CL could all increase the yield of rosmarinic acid and reduce the accumulation of caffeic acid. Therefore, L-tyrosine deaminase was heterologously expressed based on the removal of feedback inhibition of tyrosine and optimization of selectivity of 4CL to combine the advantages of Example 3 and Example 4. pACYCDuet-FjTAL-RBS-HpaBC-At4CL1 and pETDuet-aroG^{fbr}-tyrC-HPPR-CbRAS constructed in Example 4, and pCDFDuet-TcTAL-PmLAAD constructed in Example 3 were transformed into E. coli BL21(DE3) ΔtyrRΔcrrΔptsGΔpheA to obtain an engineering strain RA09. The engineering strain RA09 was cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 72 h to synthesize rosmarinic acid. As shown in FIG. 3, in the fermentation broth of RA09, the yield of rosmarinic acid was 531.7 mg/L, no accumulation of L-DOPA was detected, and the yield of caffeic acid was 141.3 mg/L.

### Example 6: Optimization of Stability of Caffeoyl Coenzyme A

In order to improve the stability of caffeoyl coenzyme A, the endogenous encoding thioesterase gene menl in E. coli was knocked out. Using the E. coli BL21(DE3) genome as a template, an upstream homologous arm U1 and a downstream homologous arm D1 of the gene menl were amplified respectively by using primer pairs F10/R10 and F11/R11, and the fragments were purified. Using the purified fragments U1 and D1 as templates, amplification was carried out by using a primer pair F10/R11 to obtain a knockout kit UD1, and the fragment was purified. In order to obtain pTarget-menl for knocking out menl, using pTarget as a template, amplification was carried out by using a primer pair F12/R12, and the fragment was purified. The purified fragment was transformed into E. coli JM109. Plasmid extraction was carried out, and sequencing was carried out for verification, thereby obtaining the correct recombinant vector pTarget-menl.

In order to produce pCas9-containing E. coli BL21 (DE3) ΔtyrRΔcrrΔptsGΔpheA electroporation-competent cells, the pCas9 plasmid was transformed into E. coli BL21 (DE3) ΔtyrRΔcrrΔptsGΔpheA chemically competent cells. The transformed monoclonal cells were picked and inoculated into 4 mL of LB medium, kanamycin with a final concentration of 50 μg/mL was added, and the cells were cultured at 30° C. for 12 h. The bacterial solution was inoculated into 50 mL of LB medium according to an inoculation amount of 2%, and a solution of kanamycin with a final concentration of 50 μg/mL and 10 mM arabinose were added. The cells were cultured at 30° C. at 220 r/min for 4 h-6 h until OD reached 0.6. The bacterial solution was transferred into a 50 ml centrifuge tube, and allowed to stand on ice for 15 min. The centrifuge tube was centrifuged at 4000 rpm at 4° C. for 10 min, and the supernatant was removed. 10 mL of 10% glycerol was added for resuspension. The operation was repeated twice, and the bacterial solution was divided according to 100 μL/package, and stored at −80° C. 400 ng of recombinant vector pTarget-menl and 1200 ng of knockout kit UD1 were added to the E. coli BL21 (DE3) ΔtyrRΔcrrΔptsGΔpheA electroporation-competent cells. The suspension was allowed to stand on ice for 10 min, and then transferred into a 1 mm electroporation cuvette that had been precooled for 10 min, and electroporation was carried out under a voltage of 1.8 kv. After the electroporation was completed, 1 ml of LB liquid medium was added, and the cells were cultured at 30° C. for 1.5 h. Colony PCR was carried out by using a primer pair F13/R13 for verification. pTarget-menl and pCas9 were dropped out from the correct monoclonal cells according to the method in the literature to obtain an engineering strain E. coli BL21 (DE3) ΔtyrRΔcrrΔptsGΔpheAΔmenl. The recombinant vectors pACYCDuet-FjTAL-RBS-HpaBC-At4CL1, and pCDFDuet-TcTAL-PmLAAD and pETDuet-aroG fb r-tyrC-HPPR-CbRAS constructed in Example 3 were transformed into the E. coli BL21(DE3) ΔtyrRΔcrrΔptsGΔpheAΔmenl to obtain an engineering strain RA10. The engineering strain RA10 was cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 72 h to synthesize rosmarinic acid. As shown in FIG. 3, in the fermentation broth of RA10, the yield of rosmarinic acid was 373.2 mg/L, the yield of caffeic acid was 33.8 mg/L, and L-DOPA was not detected.

All primer sequences are listed in Table 6.

TABLE 6
Primer sequence

| Primer | | SEQ ID |
|---|---|---|
| Name | Primer Sequence | NUMBER |
| F10 | CTCATAAAGCTAACCCGCCGTTTT | SEQ ID NO. 51 |
| R10 | CGTTGTCACCAGAAAAGTGTGACG | SEQ ID NO. 52 |
| F11 | CACACTTTTCTGGTGACAACGTCATTTAATAATCTCCAGTAAAGCCTGCACAG | SEQ ID NO. 53 |
| R11 | TACTTTGTTATCGCGATGAATATAAACTGGCACT | SEQ ID NO. 54 |
| F12 | TTGAAATCTTCGATGAGAAAGTTTTAGAGCTAGAAATAGCAAGTT | SEQ ID NO. 55 |
| R12 | TTTCTCATCGAAGATTTCAAACTAGTATTATACCTAGGACTGAGC | SEQ ID NO. 56 |
| F13 | GTGCAGCGTTCAGAAATAAGAAAACCC | SEQ ID NO. 57 |
| R13 | CCAAATGGCAAAGCCCAGCATAT | SEQ ID NO. 58 |

### Example 7: Increase of Synthesis Throughput of Salvianic Acid A to Increase Yield of Rosmarinic Acid

In order to reduce the accumulation of caffeic acid and further increase the synthesis throughput of salvianic acid A, the effects of ldh (the nucleotide sequence shown in SEQ ID NO.11) derived from L. plantarum and HPPR (the nucleotide sequence shown in SEQ ID NO.4) derived from C. scutellarioides on synthesis of rosmarinic acid were compared. Using the synthesized HPPR as a template, amplification was carried out by using a primer pair F14/R14, and the produce was purified and recovered. Using the recombinant vector pETDuet-aroG^{fbr}-tyrC-HPPR-CbRAS constructed in Example 4 as a template, amplification was carried out by using a primer pair F15/R15, and a pETDuet-aroG^{fbr}-tyrC-HPPR-CbRAS skeleton fragment was recovered. The fragment ldh and the vector pETDuet-aroG^{fbr}-tyrC-HPPR-CbRAS skeleton were recombined by Gibson assembly to obtain a recombinant vector, and the recombinant vector was transformed into E. coli JM109. Plasmid extraction was carried out, and sequencing was carried out for verification, thereby obtaining a correct recombinant vector pETDuet-aroG^{fbr}-tyrC-ldh-CbRAS. The recombinant vectors pACYCDuet-FjTAL-RBS-HpaBC-At4CL1 constructed in Example 4, and pCDFDuet-TcTAL-PmLAAD and pETDuet-aroG^{fbr}-tyrC-ldh-CbRAS constructed in Example 3 were transformed into the E. coli BL21(DE3) ΔtyrRΔcrrΔptsGΔpheAΔmenl to obtain an engineering strain RA15. The engineering strain RA15 was cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min, IPTG with a final concentration of 0.1 mM was added, and induction and fermentation were carried out at 30° C. at 220 r/min for 72 h to synthesize rosmarinic acid. As shown in FIG. 3, in the fermentation broth of RA15, the yield of rosmarinic acid was 511.2 mg/L, the yield of caffeic acid was 33.1 mg/L, and L-DOPA was not detected.

All primer sequences are listed in Table 7.

TABLE 7
Primer sequence

| Primer | | SEQ ID |
|---|---|---|
| Name | Primer Sequence | NUMBER |
| F14 | TTAAGTATAAGAAGGAGATATACATATGAAAATCATCGCGTACGCGG | SEQ ID NO. 59 |
| R14 | CATGGTATATCTCCTTTTAATCGAATTTAACCTGGGTATCCGC | SEQ ID NO. 60 |
| F15 | CATATGTATATCTCCTTCTTATACTTAACT | SEQ ID NO. 61 |
| R15 | TAAAAGGAGATATACCATGAAAATTGAAGTTAAAGATTCAACTATGATTA | SEQ ID NO. 62 |
| | AGC | |

Although the present disclosure has been disclosed as above by way of the preferred examples, it is not intended to limit the present disclosure. Anyone familiar with the art can make various changes and modifications without departing from the spirit and scope of the present disclosure. Therefore, the protection scope of the present disclosure should be as defined in the claims.

What is claimed is:

1. A recombinant Escherichia coli for synthesizing rosmarinic acid, expressing tyrosine ammonia-lyase derived from Flavobacterium johnsoniae, 4-hydroxyphenylacetate-3-monooxygenase derived from E. coli, a 3-deoxy-D-arabino-heptulosonate-7-phosphate synthase mutant, rosmarinic acid synthase derived from Coleus blumei, hydroxyphenylpyruvate reductase derived from Coleus scutellarioides, and 4-coumarate:coenzyme A ligase; wherein the 4-coumarate:coenzyme A ligase is derived from Petroselinum crispum and Arabidopsis thaliana.

2. The recombinant E. coli according to claim 1, further expressing tyrosine ammonia-lyase derived from Trichosporon cutaneum.

3. The recombinant E. coli according to claim 2, wherein the recombinant E. coli expresses the tyrosine ammonia-lyase derived from T. cutaneum, and expresses L-amino acid deaminase derived from Proteus myxofaciens.

4. The recombinant E. coli according to claim 1, wherein the hydroxyphenylpyruvate reductase gene HPPR derived from C. scutellarioides is replaced with a lactate dehydrogenase gene ldh derived from Lactiplantibacillus plantarum.

5. The recombinant E. coli according to claim 3, wherein the hydroxyphenylpyruvate reductase gene HPPR derived from C. scutellarioides is replaced with a lactate dehydrogenase gene ldh derived from L. plantarum.

6. The recombinant E. coli according to claim 5, having an endogenous thioesterase encoding gene knocked out, and expressing chorismate mutase tyrC derived from Zymomonas mobilis.

7. The recombinant E. coli according to claim 1, wherein the recombinant E. coli uses E. coli BL21(DE3) ΔtyrRΔcrrΔptsGΔpheA with tyrR, crr, ptsG and pheA genes knocked out as an original strain.

8. The recombinant E. coli according to claim 6, wherein the recombinant E. coli uses E. coli BL21(DE3) ΔtyrRΔcrrΔptsGΔpheA with tyrR, crr, ptsG and pheA genes knocked out as an original strain.

9. The recombinant E. coli according to claim 1, using E. coli BL21(DE3) ΔtyrRΔcrrΔptsGΔpheA with tyrR, crr, ptsG and pheA genes knocked out as an original strain, wherein
pETDuet-1 is used as an expression vector to express an aroG^{fbr} gene, an HPPR gene and a CbRAS gene;
pACYCDuet-1 is used as an expression vector to express FjTAL, hpaBC and At4CL1 genes; and
pCDFDuet-1 is used as an expression vector to express a TcTAL gene and a PmLAAD gene.

10. The recombinant E. coli according to claim 1, using E. coli BL21(DE3) ΔtyrRΔcrrΔptsGΔpheA with tyrR, crr, ptsG and pheA genes knocked out as an original strain; wherein
pETDuet-1 is used as an expression vector to express an aroG^{fbr} gene, an HPPR gene and a CbRAS gene;
pACYCDuet-1 is used as an expression vector to express FjTAL, hpaBC and Pc4CL1 genes; and
pCDFDuet-1 is used as an expression vector to express a TcTAL gene and a PmLAAD gene.

11. A method for producing rosmarinic acid, comprising: inoculating the recombinant E. coli according to claim 1 into a fermentation system, culturing the recombinant E. coli for a period of time, adding IPTG, and carrying out fermentation for 24-72 hours.

12. The method according to claim 11, wherein the fermentation system contains: glucose, glycerol, (NH₄)₂SO₄, K₂HPO₄·3H₂O, KH₂PO₄, MgSO₄·7H₂O, sodium citrate, vitamin B1, yeast extract, vitamin C and betaine.
